(19) United States
(12) Patent Application Publication
(10) Pub. No.: US 2025/0291954 A1
(43) Pub. Date: Sep. 18, 2025

(54) FEATURE EXTRACTION FROM MEDICAL DATA, ANONYMIZATION OF MEDICAL DATA AND PROCESSING AND/ OR ANALYSIS OF MEDICAL DATA

(71) Applicant: Siemens Healthineers AG, Forchheim (DE)

(72) Inventors: Christopher SYBEN, Cadolzburg (DE); Christian HUEMMER, Lichtenfels (DE); Dominik ECKERT, Fuerth (DE)

(73) Assignee: Siemens Healthineers AG, Forchheim (DE)

(21) Appl. No.: 19/081,274

(22) Filed: Mar. 17, 2025

(30) Foreign Application Priority Data
Mar. 18, 2024 (DE) 10 2024 202 512.7

(51) Int. Cl. G06F 21/62; G06V 10/77; G06V 10/774; G06V 10/776; G16H 50/70

(52) U.S. Cl. CPC G06F 21/6254; G06V 10/7715; G06V 10/774; G06V 10/776; G16H 50/70; G06V 2201/03

(57) ABSTRACT

A fundamental machine learning model, fMLM, is provisioned in the untrained or in a partially trained state to provide a trained machine learning model for feature extraction, xMLM, from medical data, wherein the fMLM has an architecture that is trainable by way of unsupervised or self-supervised training. The fMLM has the xMLM and at least one downstream machine learning model, nMLM, for performing at least one corresponding downstream task. First medical data is obtained and the fMLM is trained in an unsupervised or a self-supervised manner based on the first medical data. The xMLM is taken from trained fMLM and stored.

### CROSS-REFERENCE TO RELATED APPLICATION(S)

The present application claims priority under 3 5U.S.C. § 119 to German Patent Application No. 10 2024 202 512.7, filed Mar. 18, 2024, the entire contents of which is incorporated herein by reference.

### FIELD

One or more embodiments of the present invention relate to a method for providing a trained machine learning model for feature extraction from medical data. One or more embodiments of the present invention also relate to a method for anonymizing medical data that encompasses personal data, a method for providing a trained machine learning model for processing and/or analyzing medical data, and a method for processing and/or analyzing medical data. One or more embodiments of the present invention further relate to a corresponding infrastructure system and a non-transitory computer program product.

### BACKGROUND

Trained machine learning models (MLMs), in particular artificial neural networks (ANNs), have broad applications in a medical context, inter alia in a medical imaging context. In particular, such MLMs are used for classifying anatomical images, for identifying medical anomalies, for segmenting medical images, and for image preparation.

The publication by O. Ronneberger et al.: “U-Net: Convolutional Networks for Biomedical Image Segmentation” (arXiv:1505.04597) describes the U-Net architecture, a widespread CNN architecture for segmenting images that can, however, also be used for other tasks, in particular tasks relating to image-to-image translation or artifact reduction etc.

ResNet is a known architecture for artificial neural networks (ANNs), in particular convolutional neural networks (CNNs), that is described in the publication by K. He et al.: “Deep Residual Learning for Image Recognition”, Proceedings of the IEEE Conference on Computer Vision and Pattern Recognition (CVPR), 2016, pp. 770-778. Due to their ability to train very deep neural networks, ResNet models are frequently used for deep learning tasks.

Especially in the medical field, there are numerous challenges associated with the development and training of MLMs. For example, training a deep neural network with millions of parameters requires a large volume of training data in order to identify a parameter set that performs well. The generalizability of the identified parameters depends inter alia on the volume of training data used, the augmentation of the training data, and the distribution underlying the training data, i.e., the specific selection of training data.

Some approaches attempt to manage this problem by using pretrained MLMs, for example ImageNet in the field of computer vision, or use large volumes of book text and strong augmentation or text publicly available on the internet public to train large language models.

In the medical context, there are numerous known MLM architectures that can be trained in unsupervised or self-supervised manner. In some cases, a pretrained model, which may for example also have been pretrained in supervised manner, is the starting point.

The publication by F. C. Ghesu et al.: “Self-supervised Learning from 100 Million Medical Images” (arXiv:2201.01283), for example, proposed a method for self-supervised learning from large volumes of image features that is based on contrastive learning and online feature clustering. Large training data sets with over 100 million medical images from various imaging modalities, including X-ray captures, computed tomography data, magnetic resonance tomography data, and ultrasound data, are used for this purpose.

The publication by A. Dosovitskiy et al.: “An Image is Worth 16×16 Words: Transformers for Image Recognition at Scale” (arXiv:2010.11929) proposes the Vision Transformer architecture that makes the concept of neural transformer networks applicable to image data.

The publication by A. Kirillov et al.: “Segment Anything” (arXiv:2304.02643) presents the Segment Anything (SA) project for image segmentation.

The publication by Y. Zhang et al.: “Meta-Transformer: A Unified Framework for Multimodal Learning” (arXiv:2307.10802) describes a network known as Meta-Transformer for processing multimodal data, for example natural language, 2D images, 3D point clouds, audio data, video data, time series, or tabular data. Meta-Transformer uses a frozen encoder to perform multimodal perception tasks without paired multimodal training data.

The publication by A. Ramesh et al.: “Hierarchical Text-Conditional Image Generation with CLIP Latents” describes a two-stage model using a prior that generates a CLIP image embedding with the assistance of a text label, and a decoder that generates an image conditioned on the image embedding.

Autoencoders, as described for example in the publication by G. Hinton, R. Salakhutdinov: “Reducing the Dimensionality of Data with Neural Networks”, Science 313, 504-507 (2006), are multilayer artificial neural networks with a small central layer that are capable of converting high-dimensional data to low-dimensional encodings and performing reconstruction. The architecture of an autoencoder consists of an encoder module followed by a decoding module. The encoder module maps the input onto a hidden representation, also known as a latent representation or representation in latent space, for example through a plurality of fully connected layers. The decoder module maps the hidden representation, for example again through fully connected layers, back onto the original input space. The autoencoder is in particular trained in unsupervised manner.

Pretrained MLMs, for example also from the computer vision field, are also helpful, but generally insufficient, for applications in the medical field. This is inter alia because the data typically used for applications in the computer vision field differ from image data in the medical field, both with regard to image content and the nature of the image data.

For various reasons, there is very limited availability of medical data for many tasks in the medical field. For example, data protection provisions arising from official guidelines and legislation may make the use of large volumes of data outside hospitals difficult, troublesome and costly. For instance, when using medical data from a plurality of hospitals, the respective, regionally applicable data protection provisions must in particular be implemented.

The preparation of medical data for training MLMs is not integrated into clinical working methods such that additional effort is required to extract the data and prepare it for use in deep learning training. This involves, for example, data anonymization, generation of annotations in the case of supervised learning, and the normalization of data across various hospitals. Data anonymization in particular is application-dependent as various downstream tasks require different quantities of additional patient information relating, for example, to gender, age, preexisting diseases etc.

In addition, training a complete MLM for each individual task is inefficient in terms of development effort and data use and may result in trained MLMs that are tailored only to a given specific downstream task.

### SUMMARY

It is an object of one or more embodiments of the present invention to overcome at least some of the stated disadvantages.

At least this object is achieved by the subject matter of the independent claim. Advantageous further developments and preferred embodiments of the method are the subject matter of the dependent claims.

One or more embodiments of the present invention are based on the concept of using unsupervised or self-supervised training to train a fundamental machine learning model, fMLM, that has an MLM for feature extraction, xMLM, on the basis of medical data, and to take the trained xMLM from the trained fMLM and store it for later use, for example for anonymizing medical data and/or for training an MLM for processing and/or analyzing medical data, sMLM.

A first aspect of embodiments of the present invention provides a method, in particular a computer-implemented method, for providing a trained MLM for feature extraction, xMLM, from medical data. In this way, a fundamental machine learning model, fMLM, is obtained in the untrained or in a partially trained state, wherein the fMLM has an architecture that is trainable by way of unsupervised or self-supervised training. The fMLM encompasses the xMLM and at least one downstream MLM, nMLM, for performing at least one corresponding downstream task, in particular medical task. First medical data is obtained and the fMLM including the xMLM and in particular the xMLM and the at least one nMLM are trained in unsupervised and/or self-supervised manner on the basis of the first medical data. The trained xMLM is stored, for example taken from the trained fMLM and stored, in particular on a computer-readable storage medium and/or as a hardware implementation.

The at least one trained nMLM can likewise be stored, in particular on a computer-readable storage medium and/or as a hardware implementation, for example together with the trained xMLM or separately therefrom.

Unless otherwise stated, all the steps of the computer-implemented method for providing the trained xMLM can be carried out by a first data processing system that encompasses at least one first data processing device. In particular, the at least one first data processing device is configured or adapted to carry out the steps of the computer-implemented method. To this end, the at least one first data processing device may for example store a computer program encompassing commands that, when executed by the at least one first data processing device, cause the at least one first data processing device to carry out the computer-implemented method. The expressions “data processing system” and “at least one data processing device” may be used interchangeably here and hereinafter. This also applies to corresponding expressions derived therefrom.

If the at least one first data processing device encompasses two or more first data processing devices, specific steps carried out by the at least one first data processing device may also be understood as meaning that different first data processing devices carry out different steps or different parts of a step. In particular, it is not necessary for each first data processing device to perform the steps. In other words, performance of the steps may be distributed between the two or more first data processing devices.

Each embodiment of the computer-implemented method for providing the trained xMLM results in a corresponding embodiment of a method that is not purely computer-implemented in that corresponding steps for producing training data for unsupervised or self-supervised training of the fMLM are included.

Generally speaking, a trained MLM can replicate cognitive functions that people associate with different human insights. In particular, the MLM may be enabled, through training on the basis of training data, to adapt to new circumstances and detect and extrapolate patterns. Another term for a trained MLM is “trained function”.

In general, the parameters of an MLM can be adapted or updated by training. Supervised training, semi-supervised training, unsupervised training, reinforcement learning and/or active learning may in particular all be used in this context. Representation learning, which is also known as feature learning, can furthermore be used. In particular, the parameters of the MLM can be iteratively adapted by a plurality of training steps. In particular, a given loss function, also known as a cost function, may be minimized during training. During training of an artificial neural network (ANN), it is in particular possible to use the backpropagation algorithm.

An MLM may in particular encompass an ANN, a support vector machine, a decision tree and/or a Bayesian network and/or the MLM may be based on k-means clustering, Q-learning, genetic algorithms and/or association rules. In particular, an ANN may be or encompass a deep neural network, a convolutional neural network (CNN) or a convolutional deep neural network. Moreover, an ANN may be an adversarial network, a deep adversarial network and/or a generative adversarial network.

Where an MLM or part of an MLM is obtained, it may be understood here and hereinafter, for example, to mean that it is obtained in computer-readable form, for example stored on a data storage medium. Unless otherwise stated, the process of obtainment in particular does not include any further method steps for producing the MLM or the part of the MLM, such as for instance steps for training the MLM or the part of the MLM.

The fact that the fMLM is obtained in the untrained state may be understood to mean that only the architecture of the fMLM is predetermined or the fMLM is provided merely in the initialized state. If the fMLM is obtained in the partially trained state, this may be understood to mean that the fMLM is provided pretrained, wherein the pretraining may be supervised, unsupervised and/or self-supervised but, unless otherwise stated, is not part of the method according to embodiments of the present invention. The pretraining may likewise be based on medical data and/or on other data. The fMLM may be a known MLM architecture, in particular a known ANN architecture. It is also possible that, although the xMLM and the at least one nMLM are known MLM architectures, the combination of the xMLM and the at least one nMLM is not already known. In both cases, the unsupervised and/or self-supervised training may be performed with the assistance of known training methods, in particular using known loss functions.

In particular, ANNs such as for instance the above-mentioned autoencoders, U-Net, ResNet, Segment Anything, Vision Transformer, Meta-Transformer, other transformer networks, and those described in the publications by Ramesh et al. and Ghesu et al. may be considered for the xMLM and the at least one nMLM.

In the case of supervised training (also known as supervised learning), the training data used for training purposes is manually, automatically, or partly automatically provided with annotations, also known as labels that represent the ground truth. The loss functions used may then effectively compare the respective prediction of the MLM to the trained with the corresponding ground truth and the MLM can be adapted on the basis of the result.

In the case of unsupervised training (also known as unsupervised learning), the training or adaptations of the MLM to be trained is/are performed without annotated training data, i.e., without a previously known ground truth and without a reward from the environment, as is used in reinforcement learning.

The expression self-supervised training (also known as self-supervised learning) is not used entirely uniformly in the relevant literature. Here and hereinafter, the expression “self-supervised training” is used in a way that encompasses training methods that correspond to neither supervised nor unsupervised training but in which, as with unsupervised training, no annotations of the training data, i.e., no previously known ground truth, are needed. This in particular includes training methods in which the MLM to be trained and/or an auxiliary MLM generates annotations or effective annotations, in particular without human involvement. Training methods are also included in which training data is intentionally modified, in particular degraded, masked, made noisy etc., and the MLM to be trained is adapted such that it reconstructs the unmodified training data, such as for instance in the case of an ANN of the autoencoder category.

By using the fMLM on input data, the latter is converted in particular by way of the xMLM into encoded features, sometimes also known as embeddings or as latent features or features in the latent space of the fMLM. The at least one nMLM is applied in each case to the encoded features to perform a respective predefined task. Depending on the architecture of the fMLM, the xMLM may therefore also be denoted an encoder module or embedding module. With some fMLM architectures, the at least one nMLM may also be denoted as least one decoder module.

The tasks assigned to the at least one nMLM may vary in nature. In particular, it is not necessary for these tasks to correspond to a later purpose of the xMLM or to the purpose of features extracted therewith, but this is possible in principle. A task of an nMLM may, for example, comprise maximally exact reconstruction of the input data, a segmenting task, a classifying task, an object recognition task etc. These tasks are here also denoted downstream tasks since they take place downstream of feature extraction by the xMLM.

In the case of medical data, said data may vary in type and have been generated in medical or clinical contexts, in particular in a clinic or hospital, for one or more specific people, in particular patients. Medical data may in particular encompass image data, for instance two-dimensional image data or three-dimensional image data that may additionally, for example, be time-dependent as a further dimension. The image data may, for example, encompass X-ray projection images, raw data from X-ray detectors, reconstructions from computed tomography (CT) methods, raw data from magnetic resonance imaging (MRI) methods, preprocessed raw data from MRI methods, MRI reconstructions, image data from ultrasound imaging methods, image data from positron emission tomography (PET) methods, and/or from other medical imaging methods. The image data may also encompass planning and/or reference data for carrying out imaging methods and/or from surgical interventions or other medical treatment or diagnostic methods. Medical data may in particular also encompass text data, in particular unstructured text data, such as for instance physicians' reports, medical history data etc., and tabular or otherwise structured text data and/or numerical data, such as for instance laboratory reports, test results, patient identification data etc. In general, therefore, medical data may in particular encompass data specific to individuals and thus, for example, subject to data protection regulations.

The trained xMLM may be understood to be the result of the inventive method according to the first aspect of embodiments of the present invention. The trained and stored XMLM may in particular be used for various purposes, for example to train an MLM to process and/or analyze medical data, sMLM. The sMLM may here be one of the nMLMs of the fMLM or a further nMLM of the above-described type. To train the sMLM, the latter may be combined with the trained xMLM, such that the trained xMLM is applied to further training data in order to produce further encoded features, the sMLM then being applied to the further encoded features in order to perform a corresponding predefined task assigned to the sMLM.

Since the xMLM has already been trained using the method according to the first aspect of embodiments of the present invention, it has in particular already learned to extract the features from medical data that are most relevant in a medical context. The training effort for the sMLM may therefore be markedly reduced, in particular in terms of the volume of necessary training data and/or the number of necessary training iterations or training epochs. A further advantage is that the fMLM is trained in unsupervised or self-supervised manner, thus in particular without the need for manual annotations. The medical data arising in any case in large volumes in the clinical context, in particular during regular hospital operation, can be used to train the fMLM and thus the xMLM without it having to leave the hospital or the hospital's data processing infrastructure. There is no need for experts to prepare and annotate the medical data, nor need extra time be devoted for this purpose.

Last but not least, from a data protection standpoint no restrictions are to be expected, since the medical data can be processed where it arises, in particular in the hospital. Once the trained xMLM has been provided, it can straightforwardly be taken away from the hospital environment without any data protection concerns, for example in order to train the sMLM. To this end, large quantities of further encoded features may in particular be generated by applying the trained xMLM to second medical data. These further encoded features constitute anonymized data, since the underlying second medical data cannot be easily reconstructed therefrom, in particular not by the trained xMLM alone. The anonymized data may then be used outside the clinical infrastructure to train the sMLM without the second medical data itself being needed. This makes it possible, in particular, also to train the sMLM simply and without any data protection concerns in a research and/or development facility or the like of a business enterprise or a manufacturer of medical devices, software and solutions.

According to at least one embodiment, the fMLM is an ANN. Parts of an ANN may optionally likewise be denoted ANNs or modules of an ANN. The xMLM and the at least one nMLM in particular are ANNs. In particular, the xMLM is a deep ANN, i.e., having one or more, preferably multiple hidden layers.

According to at least one embodiment, the fMLM has an architecture that is trainable by unsupervised or self-supervised training on the basis of multimodal data, and the first medical data is first multimodal medical data.

Multimodal data may here and hereinafter be understood as encompassing two or more data records in different formats and/or from different sources, in particular imaging modalities. Data that thus for example encompasses both image data and text data is an example of multimodal data. Data that for example encompasses both X-ray projection images and MRI reconstructions is likewise an example of multimodal data. The multimodal data here relates for example at least in part to the same person.

Multimodal data very often arises in a medical context, for example image data from imaging modalities and text data from physicians' reports, tabular data from laboratory reports etc. It is therefore particularly advantageous in this case to train the fMLM on the basis of multimodal medical data, thereby using all available types of information. By combining data in different formats and/or from different sources, the individual constituents of the first medical data are contextualized, the xMLM thereby being trained by the unsupervised or self-supervised training particularly effectively to extract features of particular relevance from medical data. In other words, as described above, the quality of the features extracted by the xMLM is greatly enhanced in terms of further use in a medical context.

The above-mentioned architectures for MLMs, in particular ANNs, in particular autoencoders, Segment Anything, Vision Transformer, Meta-Transformer and those described in the publications by Ramesh et al. and Ghesu et al., for example, are suitable for processing multimodal data.

According to at least one embodiment, the first medical data encompasses first image data from an imaging method.

For example, an imaging method is carried out, in particular using an imaging modality, in order to generate at least some of the first medical data, in particular the first image data from the imaging method. In such embodiments, as mentioned above, the method is, for example, not purely computer-implemented.

The imaging method may for example be an X-ray imaging method, a CT method, an MRI method, a PET method, an ultrasound imaging method etc.

Image data from imaging methods constitutes particularly valuable and relevant information forming the basis for feature extraction in a medical context. This is above all also due on the fact that subsequent potential applications of the trained xMLM may often be based on image data from imaging methods as input data. Accordingly, the xMLM is trained by the unsupervised or self-supervised training particularly effectively to extract features of particular relevance from medical data. As described above, the quality of the features extracted by the xMLM is enhanced in terms of further use in a medical context.

The imaging modality may, for example, be configured to transfer, in particular directly, the first image data to the first data processing system once it has been generated, and the first data processing system may then, as described, also train the fMLM on the basis of at least the first image data. This may take place in particular during regular use of the imaging modality, for example also after initial training of the fMLM and/or to refine or further train the fMLM. In this way, a “lifelong learning” approach may also be implemented.

A second aspect of embodiments of the present invention provides a method, in particular a computer-implemented method, for anonymizing medical data. In this case, a method is carried out according to the first aspect of embodiments of the present invention in order to provide a trained xMLM. Second medical data, in particular second multimodal medical data, that encompasses personal data is obtained. The second medical data is anonymized by being encoded by applying the trained xMLM to the second medical data.

In other words, the second aspect of embodiments of the present invention provides use of an xMLM provided by a method according to the first aspect of embodiments of the present invention for anonymizing medical data.

Unless otherwise stated, all the steps of the computer-implemented method for anonymizing medical data may be carried out by the first data processing system or a further data processing system. The explanations relating to the computer-implemented method according to the first aspect of embodiments of the present invention apply in an analogous manner.

As output of the trained xMLM, the encoded second medical data constitutes anonymized data, since the underlying second medical data cannot be easily reconstructed therefrom, in particular not by the trained xMLM alone. The anonymized data can therefore be put to further use without data protection concerns. Further use may, as mentioned above, include training the sMLM. It is, however, also possible for the anonymized data merely to be securely transported or transferred and for the second medical data to be reconstructed again by a trustworthy location that a corresponding nMLM. The nMLM may thus be regarded as key to data reconstruction. For example, the xMLM may, together with the nMLM, in this case form an autoencoder or another MLM for data reconstruction.

According to at least one embodiment, the second medical data encompasses second image data from an imaging method.

For example, an imaging method is carried out, in order to generate at least some of the second medical data, in particular the second image data from the imaging method.

According to at least one embodiment, the personal data encompasses image data from an imaging method and/or text data relating to a medical assessment or appraisal of the patient and/or tabular data relating to a medical assessment or appraisal of the patient and/or numerical data relating to a medical assessment or appraisal of the patient and/or data relating to the patient's identity.

A third aspect of embodiments of the present invention provides a method, in particular a computer-implemented method, for providing a trained MLM for processing and/or analyzing medical data, sMLM. In this case, a method is carried out according to the second aspect of embodiments of the present invention in order to generate the encoded second medical data. The sMLM is obtained, in particular in a untrained or partially trained state. The sMLM is trained in unsupervised or self-supervised manner on the basis of the encoded second medical data.

In other words, the third aspect of embodiments of the present invention provides use of an xMLM provided by a method according to the first aspect of embodiments of the present invention for providing a trained sMLM.

Unless otherwise stated, all the steps of the computer-implemented method for providing a trained sMLM may be carried out by the first data processing system or a second data processing system. The explanations relating to the computer-implemented method according to the first aspect of embodiments of the present invention apply in an analogous manner.

In a method according to the third aspect of embodiments of the present invention, the sMLM is thus trained in particular directly on the basis of the anonymized data, i.e., the encoded second medical data, without the second medical data itself being needed again. This may proceed, for example, in such a way that the xMLM is applied to the second medical data in the hospital in order to generate the anonymized data, the anonymized data is transferred from the hospital to another location, for instance a research and/or development facility or a service enterprise etc., and the sMLM is trained there on the basis of the anonymized data.

The sMLM may in this case be one of the at least one nMLM of the fMLM that is used in training the fMLM, in particular the xMLM. In this case, the sMLM is provided already pretrained, for example. The sMLM may, however, also be different from the at least one nMLM, in particular a different task may be assigned to the sMLM than to the at least one nMLM.

Since the xMLM has already been trained according to the first aspect of embodiments of the present invention on the basis of the first medical data before the sMLM is trained on the basis of the encoded second medical data, very much less training data is needed to train the sMLM than in conventional approaches. This is because the xMLM is capable, even at the start of training of the sMLM, of extracting the features of particular relevance for medical applications.

It is also possible in some embodiments to use further training data, for example publicly available or conventionally anonymized data, to train the sMLM in addition to the encoded second medical data.

Per se known methods may be used to train the sMLM. For example, methods may be used such as are also used to train the fMLM, wherein the xMLM is however frozen, for example, i.e., not modified any further. It is also possible, however, for the xMLM to be further trained together with the SMLM.

According to at least one embodiment, the sMLM is trained by predicting a processing and/or analytical result on the basis of the encoded second medical data by applying the sMLM to the encoded second medical data. A predetermined loss function is evaluated as a function of the predicted processing and/or analytical result. The sMLM is updated as a function of a result of the evaluation of the loss function.

If the sMLM is an ANN, updating the sMLM in particular encompasses updating sMLM weights, in particular using an appropriate algorithm, for example a backpropagation algorithm.

According to at least one embodiment, the xMLM remains unchanged when training the sMLM on the basis of the encoded second medical data.

In other words, the xMLM is not updated as a function of the result of the evaluation of the loss function. The xMLM is thus in particular frozen when training the sMLM. It is also possible for the sMLM to be trained in the absence of the xMLM if an appropriate volume of second medical data is initially anonymized by the xMLM and provided for training the SMLM.

In this way, the training effort involved in training the sMLM may be further reduced.

According to at least one embodiment, training of the sMLM is carried out on the basis of the encoded second medical data by way of the second data processing system that has no access to the second medical data.

Advantageously, it is accordingly possible to generate and anonymize the second medical data inside a hospital or other secure area and to train the sMLM outside the hospital or secure area without concerns regarding data protection of the second medical data.

Ensuring that the second data processing system does not have access to the second medical data may be achieved, for example, by a hard separation of the second data processing system from a storage device on which the second medical data is stored, or indeed by securing the second medical data through encryption or password protection or the like.

A fourth aspect of embodiments of the present invention provides a method for processing and/or analyzing medical data. In this case, a method is carried out according to the third aspect of embodiments of the present invention in order to provide a trained sMLM. Further encoded data is obtained. Alternatively, third medical data, in particular third multimodal medical data, is obtained and the further encoded data is generated by applying the xMLM to the third medical data. A processing and/or analytical result is produced by applying the trained sMLM to the further encoded data.

In other words, the fourth aspect of embodiments of the present invention provides use of an sMLM provided by a method according to the third aspect of embodiments of the present invention for processing and/or analyzing medical data, in particular use of an xMLM provided by a method according to the first aspect of embodiments of the present invention and an sMLM provided according to the third aspect of embodiments of the present invention for processing and/or analyzing medical data.

Unless otherwise stated, all the steps of the computer-implemented method for processing and/or analyzing medical data may be carried out by the first data processing system or the second data processing system or a third data processing system. The explanations relating to the computer-implemented method according to the first aspect of embodiments of the present invention apply in an analogous manner.

According to at least one embodiment, the third medical data encompasses third image data from an imaging method.

An imaging method is carried out, for example, in order to generate at least some of the third medical data, in particular the third image data from the imaging method.

According to at least one embodiment, the sMLM is an MLM for anatomical image classification. In particular, the third medical data contains further image data from an imaging method.

Output data from the sMLM may then, for example, encompass one of a number of predefined first classes of further image data and/or an object represented by the further image data. Algorithms for medical image processing are often optimized for specific anatomical content. However, in day-to-day clinical practice it is not necessarily certain that the correct protocols and thus the correct tags will be used in the DICOM headers or the like. Classification of the further image data is therefore advantageous in order to ensure correct use of the algorithms for medical image processing. The various first classes or anatomical content may, for example, specify body parts such as “hand”, “foot”, “chest” etc. or organs such as “liver”, “heart”, “brain” etc.

According to at least one embodiment, the sMLM is an MLM for identifying a disease or an anomaly, in particular a medical or anatomical anomaly. In particular, the third medical data contains further image data from an imaging method.

Output data from the sMLM may then, for example, encompass one of a number of predefined second classes of further image data and/or an object represented by the further image data and/or a position of the object in the image data. Often only very little training data is available in particular for such applications. One or more embodiments of the present invention are therefore particularly advantageous here.

According to at least one embodiment, the sMLM is an MLM for medical image segmentation. In particular, the third medical data contains further image data from an imaging method.

Output data from the sMLM may then for example encompass a segmented image in which regions or pixels of the further image data that belong to one or more predefined object classes, are appropriately tagged, or in which the one or plurality of object classes are assigned to the regions or pixels. The object classes may for example specify specific organs, vessels, bone structures, medical devices such as catheters, stents or guide wires etc. Often only very little training data is available in particular for such applications. One or more embodiments of the present invention are therefore particularly advantageous here.

According to at least one embodiment, the sMLM is an MLM for image preparation. In particular, the third medical data contains further image data from an imaging method.

Output data from the sMLM then in particular contains prepared versions of the further image data. Preparation may, for example, include noise reduction, geometric transformation, for instance rotation or correction of distortion, contrast enhancement, other digital filtering etc.

Each of the above-stated aspects of embodiments of the present invention gives rise to further embodiments of the corresponding method from the various configurations of the method according to the other aspects of embodiments of the present invention. In particular, individual features and corresponding explanations and advantages regarding the various embodiments of methods according to one of the aspects of embodiments of the present invention can be applied in an analogous manner to corresponding embodiments of the methods of the other aspects of embodiments of the present invention.

A fifth aspect of embodiments of the present invention provides an infrastructure system for carrying out a method according to the fourth aspect of embodiments of the present invention, an inventive method for providing the trained sMLM. The infrastructure system has a first data processing system that is adapted to carry out a method according to the first aspect of embodiments of the present invention for providing a trained xMLM, and a method according to the second aspect of embodiments of the present invention for generating anonymized data. The infrastructure system has a second data processing system that is adapted to train the sMLM on the basis of the anonymized data.

In the present disclosure, the expressions “data processing system” and “at least one data processing device” may be used interchangeably. A data processing device may in particular be taken to mean a data processing device that contains a processing circuit. The data processing device may thus in particular process data for carrying out computing operations. These may optionally also include operations for carrying out indexed access to a data structure, for example a look-up table (LUT), as well as a hardware-implemented data processing process.

The data processing device may in particular contain one or more computers, one or more microcontrollers and/or one or more integrated circuits, for example one or more application-specific integrated circuits (ASIC), one or more field-programmable gate-arrays (FPGA), and/or one or more systems on a chip (SoC). The data processing device may also contain one or more processors, for example one or more microprocessors, one or more central processing units (CPU), one or more graphics processing units (GPU) and/or one or more signal processors, in particular one or more digital signal processors (DSP). The data processing device unit may also encompass a physical or virtual cluster of computers or others of the stated units.

In various exemplary embodiments, the data processing device encompasses one or more hardware and/or software interfaces and/or one or more memory units.

A memory unit may be configured as a volatile data memory, for example as a dynamic random access memory (DRAM) or static random access memory (SRAM), or as a nonvolatile data memory, for example as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM) or as a phase-change random access memory (PCRAM).

According to at least one embodiment, the second data processing system has access neither to the first medical data or nor to the second medical data.

According to at least one embodiment, the first data processing system is adapted to obtain third medical data and to generate further anonymized data by applying the trained XMLM to the third medical data. The first data processing system in this case in particular stores the trained xMLM.

According to at least one embodiment, the first data processing system is adapted to produce a processing and/or analytical result by applying the trained sMLM to the further anonymized data. The first data processing system in this case in particular stores the trained sMLM.

According to at least one embodiment, the infrastructure system has a third data processing system that is adapted to produce a processing and/or analytical result by applying the trained sMLM to the further anonymized data. The third data processing system in this case in particular stores the trained sMLM.

According to at least one embodiment, the infrastructure system has a hospital that contains the first data processing system and/or third data processing system.

According to at least one embodiment, the infrastructure system has a research and/or development facility that contains the second data processing system and is spatially separate from the hospital.

A sixth aspect of embodiments of the present invention provides a computer program.

According to at least one embodiment, the computer program has commands that, when executed by at least one data processing system, cause the at least one data processing system to carry out a method for providing a trained xMLM according to the first aspect of embodiments of the present invention and/or a method for anonymizing medical data according to the second aspect of embodiments of the present invention.

According to at least one embodiment, the computer program has first commands that, when executed by a first data processing system, cause the first data processing system to carry out a method for providing a trained xMLM according to the first aspect of embodiments of the present invention and a method for anonymizing medical data according to the second aspect of embodiments of the present invention. The computer program has second commands that, when executed by a second data processing system, cause the second data processing system to train an sMLM on the basis of the anonymized data.

According to at least one embodiment, the computer program has first commands that, when executed by a first data processing system, cause the first data processing system to carry out a method for providing a trained xMLM according to the first aspect of embodiments of the present invention and a method for anonymizing medical data according to the second aspect of embodiments of the present invention. The computer program has second commands that, when executed by a second data processing system, cause the second data processing system to train an sMLM on the basis of the anonymized data. The computer program has third commands that, when executed by a third data processing system, cause the third data processing system to produce a processing and/or analytical result by applying the trained sMLM to the further anonymized data.

The commands, first commands, second commands and/or third commands, may for example in each case take the form of program code. The program code can be provided, for example, as binary code or an assembler and/or as source code of a programming language, for example C, and/or as a program script, for example Python.

According to a further aspect of embodiments of the present invention, a computer-readable storage medium is provided that stores a computer program according to embodiments of the present invention.

The computer program and the computer-readable storage medium are in each case computer program products with the commands, the first commands, second commands and/or third commands.

The inventive solution is described above and below with regard both to the claimed systems or and to the claimed methods. Features, advantages or alternative embodiments may be associated with the other claimed subjects and vice versa. In other words, the claims and embodiments relating to the systems may be enhanced by features that are described or claimed in connection with the respective methods. In this case, the functional features of the method are implemented by physical units of the system.

Furthermore, the solution according to embodiments of the present invention is described above and below in relation to methods and systems for using trained MLMs and in relation to methods and systems for providing trained MLMs. Features, advantages or alternative embodiments may be associated with the other claimed subjects and vice versa. In other words, claims and embodiments relating to the provision of a trained MLM may be enhanced by features that are described or claimed in connection with the use of a trained MLM. In particular, the data records used in the methods and systems may have the same properties and features as the corresponding data records that are used in the methods and systems to provide a trained MLM, the trained MLMs provided by the respective methods and systems being usable in the methods and systems.

Further features and combinations of features of the present invention are revealed by the figures and the description thereof and by the claims. In particular, further embodiments of the present invention need not necessarily contain all the features of one of the claims. Further embodiments of the present invention may include features or combinations of features that are not stated in the claims.

### BRIEF DESCRIPTION OF THE DRAWINGS

The present invention is explained in greater detail below with reference to specific exemplary embodiments and associated schematic drawings. Identical or functionally identical elements in the figures may be provided with the same reference signs. Descriptions of identical or functionally identical elements are not necessarily repeated with regard to different figures.

In the figures:

FIG. 1 shows a schematic representation of an exemplary embodiment of an infrastructure system according to the present invention;

FIG. 2 shows a schematic block diagram relating to an exemplary embodiment of a method according to the present invention for providing a trained MLM for feature extraction;

FIG. 3 shows a schematic block diagram relating to an exemplary embodiment of a method according to the present invention for anonymizing medical data;

FIG. 4 shows a schematic block diagram relating to an exemplary embodiment of a method according to the present invention for providing a trained MLM for processing and/or analyzing medical data or an exemplary embodiment of a method for processing and/or analyzing medical data;

FIG. 5 shows a schematic representation of an artificial neural network;

FIG. 6 shows a schematic representation of a convolutional neural network; and

FIG. 7 shows a schematic representation of a convolutional neural network according to a U-net architecture.

### DETAILED DESCTRIPTION

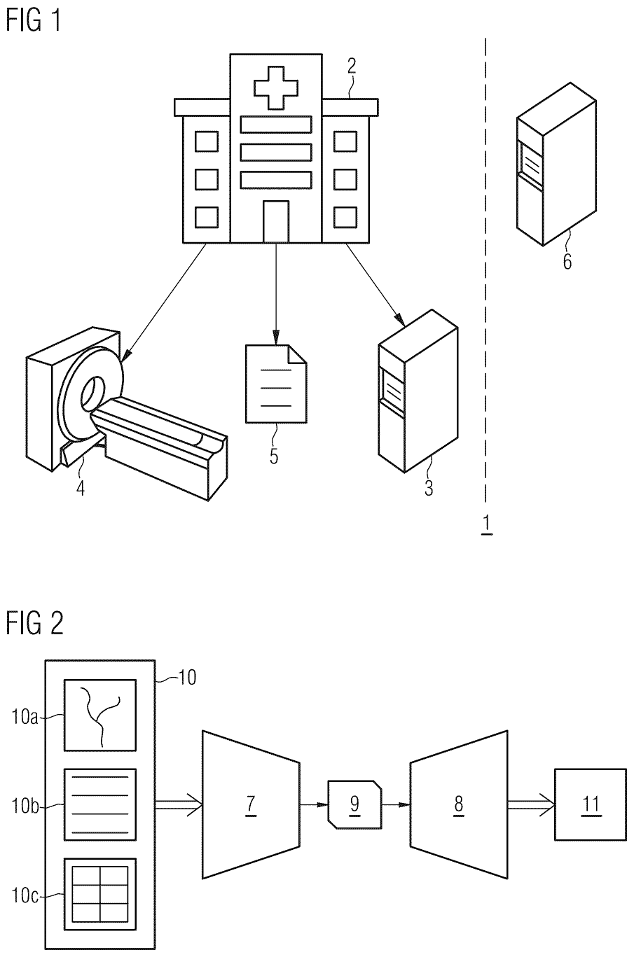

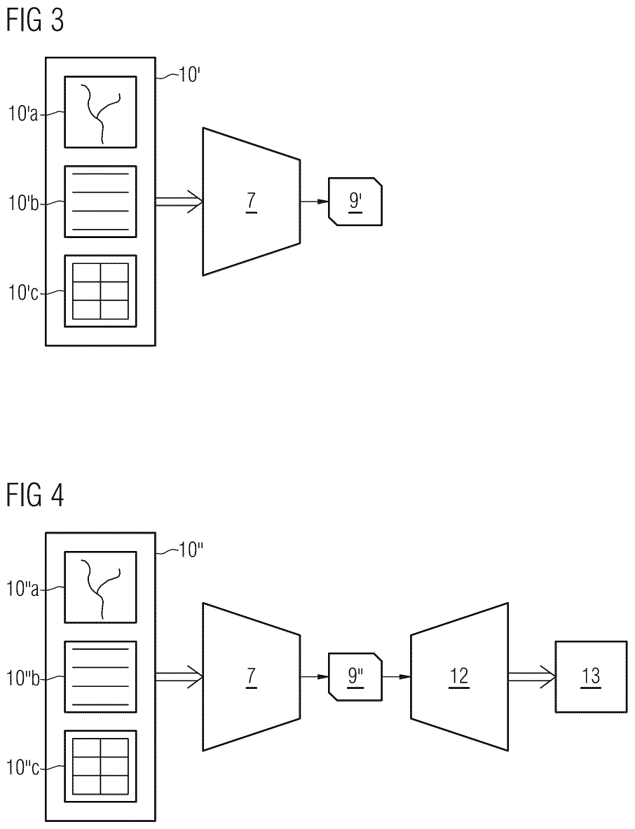

FIG. 1 shows a schematic representation of an exemplary embodiment of an infrastructure system 1 according to the present invention.

The infrastructure system 1 has a first data processing system 3 that is provided, for example, in a hospital 2. The first data processing system 3 is adapted to carry out a method for providing a trained xMLM for feature extraction, xMLM, 7 according to the first aspect of the present invention and a method for anonymizing medical data according to the second aspect of the present invention. The infrastructure system 1 has a second data processing system 6 that in particular is provided separately from the hospital 2 and that is adapted to train an MLM for processing and/or analyzing medical data, sMLM, 12 on the basis of the encoded second medical data.

FIG. 2 shows a schematic block diagram relating to an exemplary embodiment of a method according to the present invention for providing a trained xMLM 7.

To this end, a fundamental machine learning model, fMLM, 7, 8 is provided in the untrained or in a partially trained state. The fMLM 7, 8 has an architecture that is trainable by way of unsupervised or self-supervised training. The fMLM encompasses the xMLM in the untrained or a partially trained state and a downstream machine learning model, nMLM, 8 for performing a downstream task in the untrained or a partially trained state. First medical data 10 is obtained. The first medical data 10 may, for example, encompass image data 10a of one or more imaging modalities 4 of the hospital 2 and/or text data 10b from a database 5 of the hospital 2 and/or tabular data 10c from the database 5.

The fMLM 7, 8 including the xMLM 7 and the nMLM 8 is trained in unsupervised or self-supervised manner on the basis of the first medical data 10 and the trained xMLM 7 is stored, for example on a storage device of the first data processing system 3.

The xMLM 7 may, for example, encode the first medical data 10 and the nMLM 8 may reconstruct the first medical data 10 on the basis of the encoded first medical data 9. A loss function may be evaluated on the basis of the reconstructed first medical data 11 and the xMLM 7 and the nMLM 8 may be updated on the basis thereof.

FIG. 2 shows a schematic block diagram relating to an exemplary embodiment of a method according to the present invention for anonymizing medical data. A trained xMLM 7 is produced in this case, as explained with reference to FIG. 2. Second medical data 10′ that encompasses personal data is obtained. The personal data may, for example, encompass image data 10a′ from an imaging method and/or text data 10b′ and/or tabular data 10c′ and/or numerical data relating to a medical assessment or appraisal of the patient and/or data relating to the patient's identity.

The second medical data 10′ is anonymized by being encoded by applying the trained xMLM 7 to the second medical data 10′. The encoded second medical data 9′ may then be taken away from hospital 2 in a manner that is compliant with data protection regulations in order to be put to further use.

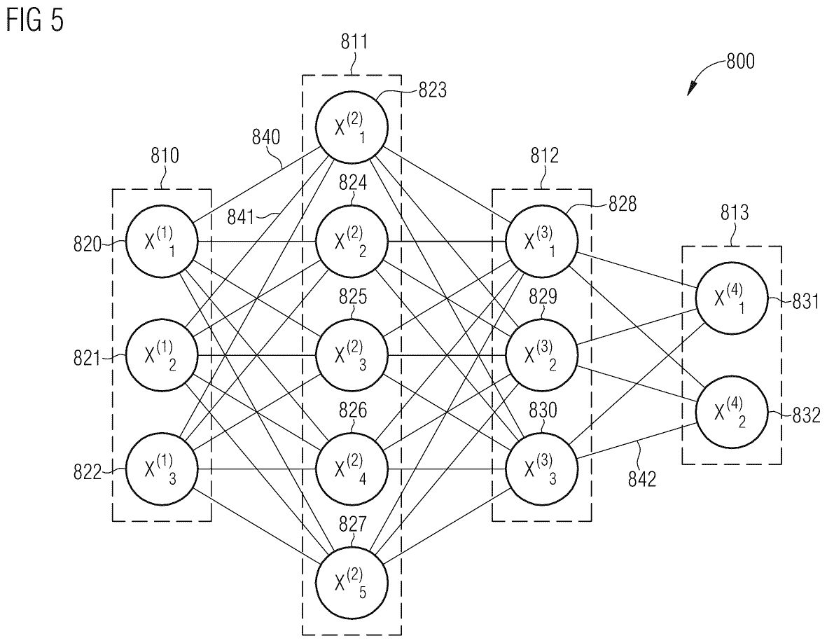

FIG. 4 shows a schematic block diagram relating to an exemplary embodiment of a method according to the present invention for providing a trained MLM for processing and/or analyzing medical data, sMLM, 12.

A trained xMLM 7 is produced in this case, as explained with reference to FIG. 2. Further medical data 10″ that encompasses personal data, for example, is obtained. The personal data may, for example, encompass image data 10a″ from an imaging method and/or text data 10b″ and/or tabular data 10c″ and/or numerical data relating to a medical assessment or appraisal of the patient and/or data relating to the patient's identity.

The further medical data 10″ is encoded by applying the trained xMLM 7 to the further medical data 10″, in particular by the first data processing system 3. The sMLM 12 is trained in unsupervised or self-supervised manner on the basis of the encoded further medical data 9″, in particular by the second data processing system 6.

The sMLM 12 is trained by predicting a processing and/or analytical result 13 on the basis of the encoded second medical data 9″ by applying the sMLM 12 to the encoded further medical data 9″. A predetermined loss function is evaluated as a function of the predicted processing and/or analytical result 13 and the sMLM 12 is updated as a function of a result of the evaluation of the loss function.

The trained sMLM 12 may then, for example, be stored on a storage device of the first data processing system 3. The first data processing system 3 may then carry out a method according to embodiments of the present invention for processing and/or analyzing medical data. To this end, third medical data is obtained, in particular from the imaging modality 4 and/or the database 5, and encoded data is obtained by applying the xMLM 7 to the third medical data. A processing and/or analytical result is produced by applying the trained SMLM 12 to the third encoded data.

According to various implementations of the aspects of embodiments of the present invention, it is proposed to use a multimodal xMLM 7 that can calculate generic embeddings, for example image embeddings, text embeddings for clinical reports, patient data etc. The xMLM 7 may, for example, be trained in unsupervised or self-supervised manner, such that a strong model for various potential tasks and adapted to the medical sector is produced that enables an sMLM 12 to be trained in a very short learning phase.

According to various implementations of the aspects of embodiments of the present invention, such a generic multimodal xMLM 7, adapted to the medical domain, can be used to process medical data. This exploits the circumstance that the embedding calculations using a deep ANN are anonymous and low-dimensional and are sharable in this form, so simultaneously complying with requirements regarding personal data protection.

According to various implementations of the aspects of embodiments of the present invention, the xMLM is trained with large volumes of data, such that it is possible to calculate generally usable embeddings that are not tied to a specific downstream task. The training data are medical data that may for example encompass image data of different imaging modalities 4 such as X-ray, MRI, CT, ultrasound etc., as well as patient data such as gender, age etc. and diagnostics data, clinical reports etc.

For training, in particular for training the xMLM, various approaches can be adopted to collect the necessary training data. “Offline learning” can be used, for example, wherein a large data set is collected and then used to train the entire data set.

On the other hand, it is additionally or alternatively possible to use “continuous learning”. This involves new data being used for further training as soon as it becomes available, without it necessarily being essential to access previously collected data, for example in the form of a large database. This also encompasses the “lifelong learning” approach that, as with human learning, involves continuous further learning on the basis of new data.

According to various implementations of the aspects of embodiments of the present invention, lifelong learning can in particular be used in order to continuously refine the fMLM, in particular the xMLM. To this end, the corresponding medical data arising in a clinical context can be directly applied and used for training and subsequently get around the clinical methods regularly provided for it. In this way, the medical data arising can be put to particularly efficient use.

According to various implementations of the aspects of embodiments of the present invention, it is, for example, possible to transmit image data generated using an imaging modality via a corresponding data transmission infrastructure to the data processing system that trains or further trains or refines the fMLM on the basis thereof, in particular in line with the lifelong learning approach.

Various implementations of the aspects of embodiments of the present invention provide for training of the sMLM to be carried out in a small number of steps, as it has only to be trained to perform the correspondingly provided task on the basis of the generic embeddings, while the already trained xMLM is either frozen or only slightly adapted to the downstream task.

According to various implementations of the aspects of embodiments of the present invention, training of the xMLM on the basis of the medical data enables good generalization and reduction of the volume of data needed to train the sMLM.

According to various implementations of the aspects of embodiments of the present invention, medical data can be anonymized by calculating the general embeddings with the trained xMLM in the hospital 2 and then be passed on for different purposes. The embeddings may be used in different frameworks for further training, for example using cloud learning, federated learning or centralized learning.

In particular, the use of low-dimensional embeddings offers various advantages, for example in respect of the reduction of the volume of data to be transmitted and the amount of computing power needed.

In particular, embeddings may be produced by the xMLM on various hardware devices, for example also on the imaging modalities 4 themselves, on PCs in hospitals, on mobile devices and/or in a cloud.

In some embodiments, examples of usable ANN architectures encompass transformer networks that may in particular be trained unsupervised for example to fill artificially generated gaps in images and text, or generative networks that can be trained unsupervised for example to produce image content from noise and text input by inverting a sequential noise addition process.

FIG. 5 shows an embodiment of an artificial neural networks (ANN) 800. The ANN 800 comprises nodes 820, . . . , 832 and edges 840, . . . , 842, wherein each edge 840, . . . , 842 is a directional connection from a first node 820, . . . , 832 to a second node 820, . . . , 832. In general, the first node 820, . . . , 832 and the second node 820, . . . , 832 are different nodes 820, . . . , 832, but it is also possible for the first node 820, . . . , 832 and second node 820, . . . , 832 to be identical. In FIG. 5, for example, the edge 840 is a directional connection from node 820 to node 823, and the edge 842 is a directional connection from node 830 to node 832. An edge 840, . . . , 842 from a first node 820, . . . , 832 to a second node 820, . . . , 832 is also designated an “incoming edge” for the second node 820, . . . , 832 and an “outgoing edge” for the first node 820, . . . , 832.

In this example, nodes 820, . . . , 832 of the ANN 800 can be arranged in layers 810, . . . , 813, wherein the layers may have an intrinsic order that is introduced by edges 840, . . . , 842 between nodes 820, . . . , 832. In particular, edges 840, . . . , 842 can only be present between adjacent layers of nodes. In the example shown, there is an input layer 810 that consists only of nodes 820, . . . , 822 without incoming edges, an output layer 813 that consists only of nodes 831, 832 without outgoing edges, and hidden layers 811, 812 between input layer 810 and output layer 813. In general, the number of hidden layers 811, 812 can be selected at will. In a multilayer perceptron (MLP) this number is at least one. The number of nodes 820, . . . , 822 within input layer 810 generally relates to the number of input values of the artificial neural network 800, and the number of nodes 831, 832 within output layer 813 generally relates to the number of output values of the artificial neural network 800.

In particular, each node 820, . . . , 832 of artificial neural network 800 can be assigned a real number as its value. In this case, x(n)i designates the value of the ith node 820, . . . , 832 of the nth layer 810, . . . , 813. The values of nodes 820, . . . , 822 of input layer 810 correspond to the input values of artificial neural network 800. The values of nodes 831, 832 of output layer 813 correspond to the output value of artificial neural network 800. Each edge 840, . . . , 842 can moreover have a weight that is a real number, in particular the weight is a real number within the range [−1, 1] or within the range [0, 1]. In this case, w(m,n)i,j designates the weight of the edge between the ith node 820, . . . , 832 of the mth layer 810, . . . , 813 and the jth node 820, . . . , 832 of the nth layer 810, . . . , 813. The abbreviation w(n)i,j is moreover defined for the weight w(n, n+1)i,j. The output values of the neural network 800 are calculated in particular by propagating the input values through the neural network 800. In particular, the values of nodes 820, . . . , 832 of the (n+1)th layer 810-813 are calculated on the basis of the values of nodes 820-832 of the nth layer 810, . . . , 813 by

$${x}_{j}^{( n + 1 )} = f ( {\sum }_{i} {x}_{i}^{( n )} {w}_{i , j}^{( n )} ) .$$

The function f therein is denoted transfer function or activation function. Known transfer functions are step functions, sigmoid functions, e.g., the logistic function, the generalized logistic function, the hyperbolic tangent, the arctangent function, the error function, the smoothstep function, or rectifier functions. The transfer function is primarily used for normalization. The values are in particular propagated layer-by-layer through the neural network 800, wherein the values of input layer 810 are provided by the input of neural network 800, wherein the values of the first hidden layer 811 can be calculated on the basis of the values of the input layer 810 of the neural network 800, wherein the values of the second hidden layer 812 can be calculated on the basis of the values of the first hidden layer 811, etc.

The neural network 800 has to be trained with training data in order to define the values w(m, n)i,j for the edges. The training data in particular comprises training input data and training output data (designated ti). In a training step, the neural network 800 is applied to the training input data in order to generate calculated output data. In particular, the training data and the calculated output data comprise a number of values that corresponds to the number of nodes of the output layer. In particular, a comparison between the calculated output data and the training data is used to recursively adapt the weights within the neural network 800 (backpropagation algorithm). In particular, the weights are modified according to the following formula

$${w}_{i , j}^{′ ( n )} = {w}_{i , j}^{( n )} - γ {δ}_{j}^{( n )} {x}_{i}^{( n )} ,$$

wherein γ is a predefined learning rate, and the numbers δ(n)j can be recursively calculated as

$${δ}_{j}^{( n )} = ( {\sum }_{k} {δ}_{k}^{( n + 1 )} {w}_{j , k}^{( n + 1 )} ) {f}^{′} ( {x}_{i}^{( n )} {w}_{i , j}^{( n )} )$$

on the basis of δ(n+1)j, if the (n+1)th layer is not the output layer 813, and

$${δ}_{j}^{( n )} = ( {x}_{j}^{( n + 1 )} - {t}_{j}^{( n + 1 )} ) {f}^{′} ( {x}_{i}^{( n )} {w}_{i , j}^{( n )} ) ,$$

if the (n+1)th layer is the output layer 813, wherein f′ is the first derivative of the activation function and t(n+1)j is the comparison training value for the jth node of output layer 813.

A convolutional neural network (CNN) is an ANN that uses a convolution operation instead a general matrix multiplication in at least one of its layers. These layers are denoted convolutional layers. In particular, a convolutional layer carries out a dot product between one or more convolutional kernels and the input data of the convolutional layer, wherein the inputs of the one or more convolutional kernels are parameters or weights that can be adapted by training. In particular, the Frobenius inner product and the ReLU activation function may be used. A convolutional neural network may comprise additional layers, for example pooling layers, fully connected layers and/or normalization layers.

Use of convolutional neural networks enables very efficient input processing, since a convolution operation based on different kernels can extract different image features, such that the relevant image features can be determined during training by adapting the weights of the convolution kernels. Moreover, combined use of the weights in the convolutional kernels means fewer parameters have to be trained, so preventing overadaptation in the training phase and enabling faster training or more layers in the network, thereby improving network performance.

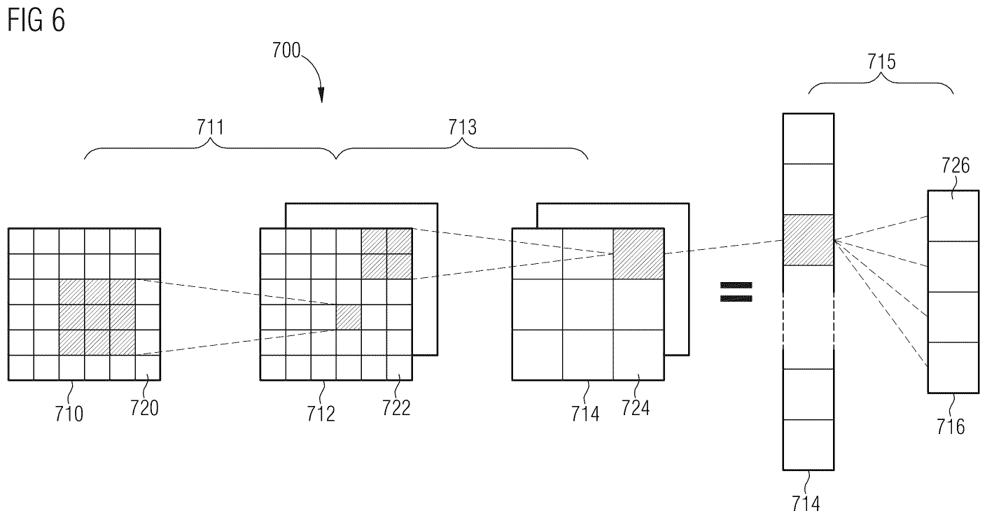

FIG. 6 shows an exemplary embodiment of a

convolutional neural network 700. In the exemplary embodiment shown, the convolutional neural network 700 comprises an input node layer 710, a convolutional layer 711, a pooling layer 713, a fully connected layer 714 and an output node layer 716, together with hidden node layers 712, 714. The convolutional neural network 700 can alternatively also comprise a plurality of convolutional layers 711, a plurality of pooling layers 713 and a plurality of fully connected layers 715 together with other kinds of layers. The order of the layers can be selected at will; fully connected layers 716 are generally used as the final layers before the output layer 715.

In particular, in a convolutional neural network 700, the nodes 720, 722, 724 of a node layer 710, 712, 714 can be regarded as a d-dimensional matrix or as a d-dimensional image. In particular, in the two-dimensional case the value of the node 720, 722, 724 indicated with i and j in the nth node layer 710, 712, 714 can be designated x(n)[i,j]. However, the arrangement of nodes 720, 722, 724 of a node layer 710, 712, 714 as such has no effect on the calculations carried out within the convolutional neural network 700, as these result solely from the structure and weights of the edges.

A convolutional layer 711 is a connecting layer between a front node layer 710 with node values x(n−1) and a rear node layer 712 with node values x(n). A convolutional layer 711 is in particular characterized by the structure and weights of the incoming edges that form a convolution operation on the basic principle of a specific number of kernels. In particular, the structure and weights of the edges of the convolutional layer 711 are selected such that the values x(n) of the nodes 722 of the rear node layer 712 are calculated as a convolution x(n)=K*x(n−1) on the basis of the values x(n−1) of the nodes 720 of the front node layer 710, wherein the convolution * is defined, in the two-dimensional case, as

$${x}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

The kernel K is here a d-dimensional matrix, in the present example a two-dimensional matrix, that is generally small in comparison with the number of nodes 720, 722, for example a 3×3 matrix or a 5×5 matrix. This means in particular that the weights of the edges in the convolutional layer 711 are not independent but rather are selected such that they provide said convolution equation. In particular, there are just 9 independent weights for a kernel in the form of a 3×3 matrix, wherein each entry of the kernel matrix corresponds to an independent weight, independent of the number of nodes 720, 722 in the front node layer 710 and the rear node layer 712.

In general, convolutional neural networks 700 use node layers 710, 712, 714 with a plurality of channels, in particular due to the use of a plurality of kernels in the convolutional layers 711. In these cases, the node layers may be regarded as (d+1)-dimensional matrices, wherein the first dimension indicates the channels. The effect of a convolutional layer 711 is then defined in a two-dimensional example as

$${x}_{b}^{( n )} [ i , j ] = {\sum }_{a} ( {K}_{a , b} * {x}_{a}^{( n - 1 )} [ i , j ] = {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] ,$$

wherein xₐ⁽ⁿ⁾ corresponds to the ath channel of the preceding node layer 710, x_{b}⁽ⁿ⁾ corresponds to the bth channel of the subsequent node layer 712 and K_{a,b} corresponds to one of the kernels. If a convolutional layer 711 acts on a preceding node layer 710 with A channels and outputs a subsequent node layer 712 with B channels, the result is A·B independent d-dimensional kernels K_{a,b}.

In general, activation functions are used in convolutional neural networks 700. In this embodiment, ReLU (rectified linear unit) is used, with R(z)=max (0, z), such that the effect of the convolutional layer 711 in the two-dimensional example is

$${x}_{b}^{( n )} [ i , j ] = R ( {\sum }_{a} ( {K}_{a , b} * {x}_{a}^{( n - 1 )} [ i , j ] ) = R ( {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] ) .$$

It is also possible to use other activation functions, for example ELU (Exponential Linear Unit), LeakyReLU, sigmoid, tanh, or softmax.

In the embodiment shown, the input layer 710 encompasses 36 nodes 720 that are arranged in a two-dimensional 6×6 matrix. The first hidden node layer 712 encompasses 72 nodes 722 arranged as two-dimensional 6×6 matrices, each of the two matrices being the result of convolving the values of the input layer with a 3×3 kernel within the convolutional layer 711. Equivalently, the nodes 722 of the first hidden node layer 712 can be interpreted as a three-dimensional 2×6×6 matrix, wherein the first dimension corresponds to the channel dimension.

One advantage of using convolutional layers 711 is that a spatially local correlation of the input data can be utilized by a local connectivity pattern being enforced between nodes of adjacent layers, in particular by each node only being connected to a small region of the nodes of the preceding layer.

A pooling layer 713 is a connecting layer between a preceding node layer 712 with node values x(n−1) and a subsequent node layer 714 with node values x(n). A pooling layer 713 can be characterized in particular by the structure and weights of the edges and the activation function that form a pooling operation on the basis of a nonlinear pooling function f. For example, in the two-dimensional case, the values x(n) of nodes 724 of the subsequent node layer 714 can be calculated on the basis of the values x(n−1) of the nodes 722 of preceding node layer 712 as follows

$${x}_{b}^{( n )} [ i , j ] = f ( {x}_{b}^{( n - 1 )} [ {id}_{1} , {jd}_{2} ] , … , {x}_{b}^{( n - 1 )} [ ( i + 1 ) {d}_{1} - 1 , ( j + 1 ) {d}_{2} - 1 ] ) .$$

In other words, by using a pooling layer 713, the number of nodes 722, 724 can be reduced by a number d1-d2 of neighboring nodes 722 in the preceding node layer 712 being replaced by a single node 722 in the subsequent node layer 714 that is calculated as a function of the values of the stated number of neighboring nodes. The pooling function f can in particular be the max function, the average or the L2 standard. In particular, in a pooling layer 713, the weights of the incoming edges are fixed and are not modified by training.

The advantage of using a pooling layer 713 is that the number of nodes 722, 724 and the number of parameters is reduced. This results in a reduction in computational effort in the network and control of overadaptation.

In the exemplary embodiment shown, the pooling layer 713 is a max pooling layer in which four neighboring nodes are replaced by just one node, wherein the value is the maximum of the values of the four adjacent nodes. Max pooling is applied to each d-dimensional matrix of the preceding layer; in this embodiment, max pooling is applied to each of the two-dimensional matrices, whereby the number of nodes is reduced from 72 to 18.

In general, the final layers of a convolutional neural network 700 may be fully connected layers 715. A fully connected layer 715 is a connecting layer between a preceding node layer 714 and a subsequent node layer 716. A fully connected layer 713 can be characterized in that a majority, in particular all, of the edges between the nodes 714 of the preceding node layer 714 and the nodes 716 of the subsequent node layer are present, the weight of each of these edges being individually adaptable.

In this embodiment, the nodes 724 of the preceding layer 714 of the fully connected layer 715 are represented both as two-dimensional matrices and additionally as non-contiguous nodes, shown as a line of nodes, wherein the number of nodes has been reduced for greater ease of depiction. This operation is also known as flattening. In this embodiment, the number of nodes 726 in the subsequent node layer 716 of the fully connected layer 715 is smaller than the number of nodes 724 in the preceding layer 714. Alternatively, the number of nodes 726 may also be greater or identical.

Moreover, in this embodiment, the softmax activation function is used within the fully connected layer 715. As a result of applying the softmax function, the sum of the values of all nodes 726 of the output layer 716 is equal to 1, and all values of all nodes 726 of the output layer 716 are real numbers between 0 and 1. In particular when the convolutional neural network 700 is used for categorizing input data, the values of the output layer 716 can be interpreted as the probability of the input data falling into one of the various categories.

In particular, convolutional neural networks 700 can be trained on the basis of the backpropagation algorithm. Overadaptation can be prevented by using regularization methods, for example the omission of nodes 720, . . . , 724, stochastic pooling, the use of artificial data, weight reduction on the basis of the L1 or L2 standard, or max standard limitations.

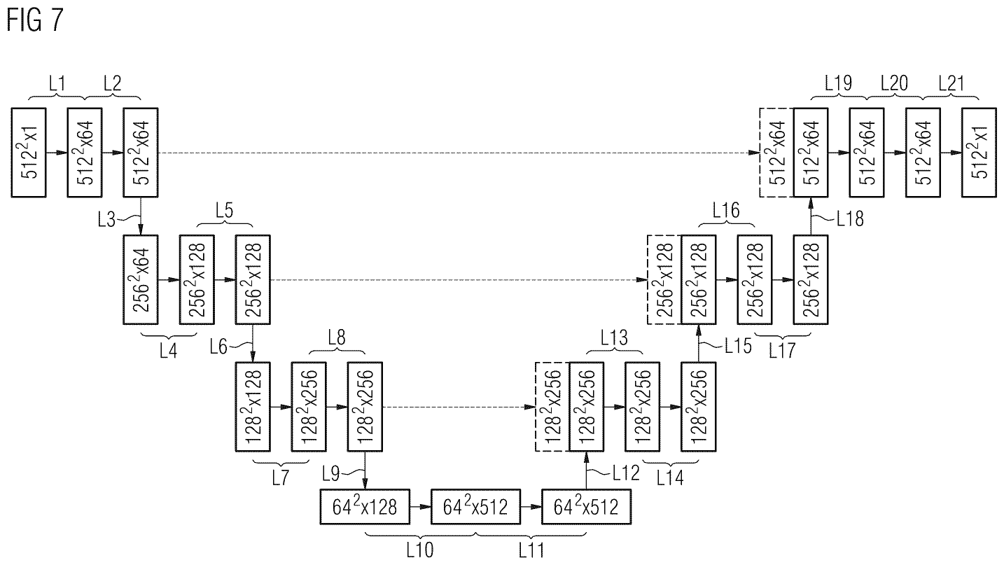

FIG. 7 is a schematic representation of a CNN with a U-Net structure. In the example shown, the input data for the CNN is a two-dimensional medical image with 512×512 pixels, each pixel encompassing an intensity value. The CNN encompasses convolutional layers shown by solid, horizontal arrows, pooling layers shown by solid arrows pointing downward, and upsampling layers shown by solid arrows pointing upward. The number of respective nodes is stated in the boxes. Within the U-Net structure, the input images are firstly downsampled, in particular by reducing the size of the images and increasing the number of channels. They are then upsampled, in particular by magnifying the images and reducing the number of channels to produce a transformed image.

All the final convolutional layers L1, L2, L4, L5, L.7, L8, L10, L11, L13, L14, L16, L17, L19, L20 apart from the final one use 3×3 kernels with a padding of 1, the ReLU activation function and a number of filters or convolutional kernels corresponding to the number of channels of the respective node layers, as shown in FIG. 6. The final convolutional layer uses a 1×1 kernel without padding and the ReLU activation function.

The pooling layers L3, L6, L9 are max pooling layers that replace four neighboring nodes with just one node, wherein the value is the maximum of the values of the four adjacent nodes. The upsampling layers L12, L15, L18 are transposed convolutional layers with 3×3 kernels and a stride of 2, so effectively quadrupling the number of nodes. The dashed horizontal arrows correspond to concatenation operations, in which the output of a convolutional layer L2, L5, L8 of the downsampling branch of the U-Net structure is used as additional inputs for a convolutional layer L13, L16, L19 of the upsampling branch of the U-Net structure. This additional input data is treated as additional channels in the input node layer for the convolutional layer L13, L16, L19 of the upsampling branch.

A database with 500 first medical images was used to train the CNN, the respective segmentation mask being prepared on the basis of annotations by specialist radiologists. In particular, the specialists defined one segmentation mask for one structure of interest for each of the 500 first medical images, wherein a value of 1 was assigned to the pixels that correspond to the structure of interest and a value of 0 was assigned to the pixels that did not correspond to the structure of interest. The database was subdivided into training data (320 data records), validation data (80 data records), and test data (100 data records). The CNN was trained using the backpropagation algorithm on the basis of a binary cross-entropy cost function

$$L ( x , y ) = {\sum }_{i} {\sum }_{j} B C E ( y [ i , j ] , M ( x ) [ i , j ] )$$

$$with$$

$$B C E ( a , b ) : = - a log ( b ) - ( 1 - a ) log ( 1 - b ) ,$$

wherein x denotes a first medical image, y defines the corresponding segmentation mask prepared by the specialist radiologists, and M(x) denotes the result of applying the CNN to the first medical input image x. Alternatively, other cost functions could also be used, such as weighted binary cross-entropy, Focal Loss or Dice Loss.

On the basis of the validation set of 80 data records and the corresponding annotations, the model with the best performance was selected from multiple machine learning models (with different hyperparameters, for example number of layers, size and number of kernels, padding etc.). Specificity and sensitivity were determined on the basis of the test set encompassing 100 data records and the corresponding annotations.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A method for providing a trained machine learning model for feature extraction from medical data, the method comprising:
obtaining a fundamental machine learning model in an untrained or a partially trained state, wherein the fundamental machine learning model has an architecture that is trainable by way of unsupervised or self-supervised training, and wherein the fundamental machine learning model encompasses a machine learning model for feature extraction and at least one downstream machine learning model to perform at least one corresponding downstream task;
obtaining first medical data;
training the fundamental machine learning model including the machine learning model for feature extraction and the at least one downstream machine learning model in an unsupervised or a self-supervised manner based on the first medical data; and
storing the trained machine learning model for feature extraction.

2. The method as claimed in claim 1, wherein the fundamental machine learning model has an architecture that is trainable by way of unsupervised or self-supervised training based on multimodal data, and wherein the first medical data is first multimodal medical data.

3. The method as claimed in claim 1, further comprising:
performing an imaging method to generate at least some of the first medical data.

4. A method for anonymizing medical data, the method comprising:
performing the method as claimed in claim 1 to provide the trained machine learning model for feature extraction;
obtaining second medical data encompassing personal data; and
anonymizing the second medical data via encoding by applying the trained machine learning model for feature extraction to the second medical data.

5. The method as claimed in claim 4, wherein the personal data includes at least one of
image data from an imaging method;
at least one of text data, tabular data or numerical data relating to a medical assessment or appraisal of a patient; or
data relating to an identity of the patient.

6. A method for providing a trained machine learning model for at least one of processing or analyzing medical data, the method comprising:
performing the method as claimed in claim 4 to generate encoded second medical data; and
training a machine learning model for at least one of processing or analyzing medical data in an unsupervised or a self-supervised manner based on the encoded second medical data.

7. The method as claimed in claim 6, further comprising:
predicting at least one of a processing result or an analytical result by applying the machine learning model for at least one of processing or analyzing medical data to the encoded second medical data;
evaluating a loss function as a function of the predicted at least one of the processing result or the analytical result; and
updating the machine learning model for at least one of processing or analyzing medical data as a function of a result of the evaluating of the loss function.

8. The method as claimed in claim 6, wherein the machine learning model for feature extraction remains unchanged when training the machine learning model for at least one of processing or analyzing medical data based on the encoded second medical data.

9. The method as claimed in claim 6, wherein the training of the machine learning model for at least one of processing or analyzing medical data is carried out based on the encoded second medical data via a data processing system that has no access to the second medical data.

10. A method for at least one of processing or analyzing medical data, the method comprising:
performing the method as claimed in claim 6 to provide a trained machine learning model for at least one of processing or analyzing medical data;
obtaining third medical data and generating encoded third medical data by applying the trained machine learning model for feature extraction to the third medical data; and
producing at least one of a processing result or an analytical result applying the trained machine learning model for at least one of processing or analyzing medical data to the encoded third medical data.

11. The method as claimed in claim 10, wherein the machine learning model for at least one of processing or analyzing medical data is a machine learning model for at least one of
anatomical image classification;
identifying a disease or an anomaly;
medical image segmentation; or
image preparation.

12. An infrastructure system for performing the method as claimed in claim 6, the infrastructure system comprising:
a first data processing system configured to provide a trained machine learning model for feature extraction; and
a second data processing system configured to train a machine learning model for at least one of processing or analyzing medical data based on the encoded second medical data.

13. The infrastructure system as claimed in claim 12, further comprising:
an imaging modality configured to perform an imaging method to generate image data, and
transfer the image data to the first data processing system, wherein
the first data processing system is configured to at least one of
use the image data to train the fundamental machine learning model, or
after training of the fundamental machine learning model, use the image data to further train the fundamental machine learning model.

14. The infrastructure system as claimed in claim 12, wherein the first data processing system is configured to
obtain third medical data, and
generate encoded third medical data by applying the trained machine learning model for feature extraction to the third medical data, and wherein
the first data processing system is configured to produce at least one of a processing result or an analytical result by applying the trained machine learning model for at least one of processing or analyzing medical data to the encoded third medical data, or
the infrastructure system includes a third data processing system that is configured to produce at least one of a processing result or an analytical result by applying the trained machine learning model for at least one of processing or analyzing medical data to the encoded third medical data.

15. The infrastructure system as claimed in claim 12, further comprising at least one of:
a hospital that contains the first data processing system; or
at least one of a research facility or a development facility that contains the second data processing system and is spatially separate from the hospital.

16. A non-transitory computer-readable medium storing computer-executable instructions that, when executed by at least one processor, cause the at least one processor to perform the method of claim 1.

17. A non-transitory computer-readable medium storing computer-executable instructions that, when executed by at least one processor, cause the at least one processor to perform the method of claim 4.

18. A non-transitory computer-readable medium storing computer-executable instructions including first commands that, when executed by at least one processor, cause the at least one processor to perform the method of claim 6.

19. The non-transitory computer-readable medium of claim 18, wherein the computer-executable instructions include second commands that, when executed by a second data processing system, cause the second data processing system to train the machine learning model for at least one of processing or analyzing medical data based on the encoded second medical data.

20. The non-transitory computer-readable medium of claim 19, wherein the computer-executable instructions include third commands that, when executed by a third data processing system, cause the third data processing system to
obtain third medical data and generate encoded third medical data by applying the trained machine learning model for feature extraction to the third medical data, and
produce at least one of a processing result or an analytical result applying the trained machine learning model for at least one of processing or analyzing medical data to the encoded third medical data.
